(19) United States
(12) Patent Application Publication
(10) Pub. No.: US 2009/0191293 A1
(43) Pub. Date: Jul. 30, 2009

(54) Composition for enhancing immunity and reducing inflammation related to infections

(75) Inventors: Marvin Heuer, (US); Ken Clement, (US); Chaudhuri Shan, (US); Megan Thomas, (US)

Correspondence Address: IOVATE HEALTH SCIENCE RESEARCH INC., 381 North Service Road West, Oakville, ON, L6M 0H4, CA

(73) Assignee: IOMEDI DEVELOPMENT INTERNATIONAL, SRL

(21) Appl. No.: 12/315,921

(22) Filed: Dec. 8, 2008

Related U.S. Application Data
(62) Division of application No. 11/733,510, filed on Apr. 10, 2007, now abandoned.

(51) Int. Cl. A61K 36/8962; A61K 36/19

(52) U.S. Cl. 424/754; 424/725

(57) ABSTRACT

A composition and method for enhancing immunity and reducing or inhibiting inflammation related innate immune system activation while affording the body of a user protection against known detrimental byproducts of immune-response is provided. The composition comprises at least a therapeutically effective amount of vitamin C and extract of Andrographis paniculata. The present invention may additionally comprise vitamin A (Retinyl palmitate), an extract of blackberry, garlic, and an extract of cranberry. A method of same is also provided.

### FIELD OF THE INVENTION

The present invention relates to immunity enhancing, anti-inflammatory compounds. More specifically, the present invention relates to a composition comprising a synergistic combination of vitamin C and plant extracts.

### BACKGROUND OF THE INVENTION

Inflammation, also called the innate immune cascade, is the result of a complex series of molecular signals involving the immune system, usually in response to infection or cellular or tissue damage. Inflammation normally constitutes the body's initiation of healing, however when it is not properly regulated inflammation can result in chronic diseases such as arthritis. On its own, inflammation is neither good nor bad for the body since inflammation does fight disease. It does, however, indirectly affect the body of an individual at a cost to normal immune and catabolic processes.

The innate cascade, or the innate immune response, is the non-specific response mounted by the immune system and is characterized by the infiltration of cells such as leukocytes such as natural killer cells, mast cells, eosinophils and basophils as well as phagocytes such as neutrophils, macrophages and dendritic cells in response to chemotatic signaling at the site of injury or infection. Molecules secreted by these aforementioned cells such as histamine and various cytokines; and the complement system of circulating proteins assist several aspects of the immune response, however contributing to inflammation through the cellular signaling for infiltration of immune cells to the cell of injury or infection. There are several common clinical symptoms associated with inflammation and the innate immune system including pain, localized redness, swelling and heat at the site of inflammation.

When inflammation persists, pro-inflammatory cytokines, such as interleukin-1 (IL-1) and tumor necrosis factor-alpha (TNF-α), are released, which act upon cell-surface receptors, causing increased migration of neutophils, monocytes, activated T-helper and T-cytotoxic cells to the area. The expression of pro-inflammatory cytokines is regulated by a group of transcription factors, belonging to the Nuclear Factor kappa B (NF-κB) family, which are found in nearly all cells (Guijarro C, Egido J. Transcription factor-kappa B (NF-kappa B) and renal disease. Kidney Int. 2001 February; 59(2):415-24). NF-κB activity is stimulated by any number of stimuli including various cytokines, stress, pathogens or chemicals.

What is currently known regarding both the upstream and down-stream NF-κB signaling is complex, however the general consensus that the NF-κB activation pathway is summarized as follows. In the unstimulated state NF-κB resides, as a dimer, in the cytoplasm sequestered by inhibitor proteins termed ‘inhibitor of kappa B’ (IkB). These IkB proteins mask the domain of NF-κB that binds to the promoter region of specific target genes, thereby blocking the ability of NF-κB to initiate transcription. Upon activation by any number of stimuli such as various cytokines, stress, pathogens or chemicals, the NF-κB portion of the dimer is liberated from IkB via the phosphorylation of IkB via IkB kinases (IKK 1 and 2) through degradation signaling of IkB. The activity of these IKK factors is essential for NF-κB activity as evidenced by the finding that mice deficient in IKK1 and IKK2 lack all NF-κB activity (Li Q, Estepa G, Memet S, Israel A, Verma I M. Complete lack of NF-κB activity in IKK1 and IKK2 double-deficient mice: additional defect in neurulation. Genes Dev. 2000 Jul. 15; 14(14):1729-33). Also, in human cell cultures, blocking the interaction of IKKgamma/NEMO with a specific peptide inhibits NF-κB activity (May M J, D'Acquisto F, Madge L A, Glockner J, Pober J S, Ghosh S. Selective inhibition of NF-κB activation by a peptide that blocks the interaction of NEMO with the IκB kinase complex Science. 2000 Sep. 1; 289(5484):1550-4), further solidifying the actions of IKK 1 and IKK 2.

The liberation of NF-κB from the dimer allows it to rapidly translocate into the nucleus and commence initiation of transcription by high affinity binding to regulatory KB motifs in target genes (Blackwell T S, Christman J W. The role of nuclear factor-kappa B in cytokine gene regulation. Am. J. Respir. Cell Mol. Biol. 1997 July; 17(1):3-9).

Although it is desirable to reduce the severity of inflammation, some inflammation will naturally occur as a result of the innate immune system mounting a defense against pathogens. During an attack on pathogens, reactive oxygen species, and other byproducts are produced which can be potentially harmful to the body. Therefore, it is desirable to afford the body protection from reactive oxygen species during an immune response.

Furthermore, it is also desirable to increase or maintain the quantity of various types of cells involved in immune responses.

A composition to inhibit inflammation and protect against harmful byproducts of an immune response is disclosed henceforth. A method is same is also presented.

### SUMMARY OF THE INVENTION

The present invention is directed towards an orally administered anti-inflammatory, immune-cell enhancing and immune-response byproduct protective composition comprising an effective amount of vitamin C and an extract of Andrographis paniculata. In additional aspects of the present invention, one or more of vitamin A (Retinyl palmitate), an extract of blackberry, garlic, ginger (dry rhizome), and an extract of cranberry are added to the composition to provide further synergistic benefits. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards an orally administrable anti-inflammatory composition, comprising an effective amount of vitamin C and a plant extract with known antioxidant properties. The present invention comprises at least vitamin C and an extract of Andrographis paniculata. According to various embodiments, the present invention may further comprise vitamin A (Retinyl palmitate), an extract of blackberry, garlic, ginger (dry rhizome), and an extract of cranberry.

### Vitamin C

Vitamin C, or L-ascorbic acid, is an essential nutrient required in small amounts in order to allow a range of essential metabolic reactions in animals and plants. The human body does not make or store vitamin C, therefore they are required to ingest it as part of their daily diests. In addition to the being a strong antioxidant and helping to protect the body from oxidative stress, Ascorbic acid is acts as a coenzyme in necessary enzymatic reactions as well as providing anti-inflammatory benefits (Wannamethee S G, Lowe G D, Rumley A, Bruckdorfer K R, Whincup P H. Associations of vitamin C status, fruit and vegetable intakes, and markers of inflammation and hemostasis. Am J Clin Nutr. 2006 Mar; 83(3):567-74).

Vitamin C concentrations in plasma and leukocytes rapidly decline during infections and stress, therefore supplementation during periods of infection is desired in order to maintain essential metabolic reactions. Furthermore, supplementation of vitamin C is known to improve antimicrobial and natural killer cell activities, in addition to increasing lymphocyte proliferation. Studies of the role of vitamin C in immune response situations have concluded that ascorbic acid helps to protect cells from reactive oxygen species that are produced during immune responses. (Wintergerst E S, Maggini S, Homig D H. Immune-enhancing role of vitamin C and zinc and effect on clinical conditions. Ann Nutr Metab. 2006; 50(2):85-94. Epub 2005 Dec. 21. Review).

In a meta-analysis it was concluded that daily supplementation of ascorbic acid, in amounts greater than 100 mg, lowers the incidence, severity and integrated morbidity of the common cold (Pauling L. The significance of the evidence about ascorbic acid and the common cold. Proc Nat Acad Sci USA 1971 November; 68(11):2678-81). It is herein understood by the inventors that the incorporation of ascorbic acid in an immune system enhancing supplement will effectively reduce incidence, severity and duration of cold related infections as well as help to maintain vitamin C levels in an individual during infections.

In an embodiment of the present invention, which is set forth in greater detail in the examples below, the immune enhancing, anti-inflammatory composition comprises vitamin C. A serving of the immune enhancing, anti-inflammatory composition comprises from about 0.100 g to about 2.000 g of vitamin C. The preferred dosage of a serving of the immune enhancing, anti-inflammatory composition comprises about 1.100 g of vitamin C.

Andrographis paniculata

Andrographis paniculata is a medicinal herb used traditionally throughout Asia used to treat a number of conditions. Clinical studies have demonstrated an immunity-enhancing benefit of Andrographis paniculata supplementation by reducing the occurrence and severity of common cold symptoms (Caceres D D, Hancke J L, Burgos R A, Sandberg F, Wikman G K. Use of visual analogue scale measurements (VAS) to assess the effectiveness of standardized Andrographis paniculata extract SHA-10 in reducing the symptoms of common cold. A randomized double blind-placebo study. Phytomedicine. 1999 October; 6(4):217-23) as well as upper-respiratory tract infections (Melchior J, Spasov A A, Ostrovskij O V, Bulanov A E, Wikman G. Double-blind, placebo-controlled pilot and phase III study of activity of standardized Andrographis paniculata Herba Nees extract fixed combination (Kan jang) in the treatment of uncomplicated upper-respiratory tract infection. Phytomedicine. 2000 October; 7(5):341-50).

One of the active components of Andrographis paniculata extracts is the diterpene, andrographolide. (Shen Y C, Chen C F, Chiou W F. Andrographolide prevents oxygen radical production by human neutrophils: possible mechanism(s) involved in its anti-inflammatory effect. Br J Pharmacol. 2002 January; 135(2):399-406). Andrographolide is known to inhibit expression of intercellular adhesion molecule-1 which is activated by tumor necrosis factor-alpha (TNF-α). Furthermore, it has been shown that andrographolide specifically affects the binding of nuclear factor kappa B (NF-κB) to the DNA, thus inhibiting pro-inflammatory cytokine expression (Hidalgo M A, Romero A, Figueroa J, Cortes P, Concha I I, Hancke J L, Burgos R A. Andrographolide interferes with binding to nuclear factor-κB to DNA in HL-60-derived neutrophilic cells. Br J Pharmacol. 2005 Mar; 144(5):680-6). Although the previously mentioned research is specific to HL-60-derived neutrophic cell, nuclear Factor kappa B (NF-κB) is a family of transcription factors found in nearly all cells and is involved with regulating immune responses (Guijarro C, Egido J. Transcription factor-kappa B (NF-kappa B) and renal disease. Kidney Int. 2001 February; 59(2):415-24). Thus, it is understood by the inventors that andrographolide will confer NF-κB inhibitory properties in several other cell types as found in the body of an individual.

It is understood by the inventors that the incorporation of an inhibitor of the NF-κB pathway in an anti-inflammatory composition will attenuate the production of pro-inflammatory cytokines, thus resulting in a reduction in relation to the intensity and duration of inflammation-related symptoms.

In an embodiment of the present invention, which is set forth in greater detail in the examples below, the immune enhancing, anti-inflammatory composition comprises Andrographis paniculata. A serving of the immune enhancing, anti-inflammatory composition comprises from about 0.0001 g to about 1.0 g of Andrographis paniculata. The preferred dosage of a serving of the immune enhancing, anti-inflammatory composition comprises about 0.001 g of Andrographis paniculata.

### Vitamin A (Retinyl Palmitate)

Retinol, the animal form of vitamin A, is a fat soluble, antioxidant that is an essential human nutrient, important for the health of the mucus lining of the nose, mouth, sinuses, and stomach. Vitamin A plays an important role in the maintenance of epithelial tissues which provide a physical barrier for the body to pathogens which can lead to infection. It is also involved in maintaining various immune cell types from both the innate and the acquired immune systems, including the lymphocytes, such as B-cells, T-cells, and natural killer cells, as well as many myelocytes such as neutrophils, macrophages, and myeloid dendritic cells.

A metabolite of vitamin A, all trans-retinoic acid (atRA), has been shown to lower the production of pro-inflammatory cytokines (e.g. IL-12 and TNF-α) while enhancing the production of the immune modulating cytokine, IL-10 (Wang X, Allen C, Ballow M. Retinoic Acid Enhances the Production of IL-10 While Reducing the Synthesis of IL-12 and TNF-alpha from LPS-Stimulated Monocytes/Macrophages. J Clin Immunol. 2007 Jan. 26; [Epub ahead of print] (Abstract)).

IL-12 is a cytokine that is naturally produced by macrophages in response to antigenic stimulation. This cytokine stimulates the production of TNF-α from both T and natural killer cells and mediates the enhancement of the cytotoxic activity of natural killer cells and cytotoxic T lymphocytes. IL-10, on the other hand, is an anti-inflammatory cytokine that inhibits the synthesis of pro-inflammatory, cytokines (interferongamma, IL-2, IL-3 and TNF-α), which are produced by macrophages and T helper cells. IL-10 is often released by cytotoxic T-cells to inhibit the action of natural killer cells during the acquired immune response to viral infections.

It is understood by the inventors that incorporation of vitamin A in an anti-inflammatory composition will attenuate the production of pro-inflammatory cytokines while at the same time increasing the production of anti-inflammatory cytokines thus resulting in a decrease in severity and duration of inflammation.

In an embodiment of the present invention, which is set forth in greater detail in the examples below, the immune enhancing, anti-inflammatory composition comprise vitamin A. A serving of the immune enhancing, anti-inflammatory composition comprises from about 1000 IU to about 10000 IU of vitamin A. The preferred dosage of a serving of the immune enhancing, anti-inflammatory composition comprises about 5010 IU of vitamin A.

### Blackberry Extract

The blackberry is a widespread and well known shrub; commonly called a bramble in the eastern United States and Europe. Blackberry contains the water-soluble flavonoid, cyanidin-3-glucoside (C3G), which has been shown to be the active compound responsible for blackberries' antioxidant benefits.

In a study of the effects of blue honeysuckle extract on lipopolysaccharide-induced (LPS), a commonly known and used initiator of innate immune responses, inflammation (Jin X H, Ohgami K, Shiratori K, Suzuki Y, Koyama Y, Yoshida K, Ilieva I, Tanaka T, Onoe K, Ohno S. Effects of blue honeysuckle (Lonicera caerulea L.) extract on lipopolysaccharide-induced inflammation in vitro and in vivo. Exp Eye Res. 2006 May; 82(5):860-7. [Epub 2005 Nov. 23] (Abstract)), C3G was shown to inhibit the NF-κB dependent signaling pathway, thereby reducing the production of pro-inflammatory mediators.

It is understood by the inventors that the incorporation of a known inhibitor of the NF-κB dependent signaling pathway in an anti-inflammatory composition will effectively reduce production of pro-inflammatory mediators. This lessening of pro-inflammatory cytokine production will result in a lowering of inflammation observed during infections or injury.

In an embodiment of the present invention, which is set forth in greater detail in the examples below, the immune enhancing, anti-inflammatory composition comprises an extract of blackberry. A serving of the immune enhancing, anti-inflammatory composition comprises from about 0.0001 g to about 1.000 g of an extract of blackberry. The preferred dosage of a serving of the immune enhancing, anti-inflammatory composition comprises about 0.0010 g of an extract of blackberry.

### Garlic

Garlic is the common name for a species of onion, Allium sativum L. Allium sativum has been found to regulate blood sugar levels and posses some cancer-fighting properties. Moreover, garlic is known to stimulate the body's immune system, particularly through enhancing the macrophages and lymphocytes. Allicin is a powerful antibiotic and anti-fungal compound that is obtained from uncooked, freshly cut garlic cloves.

A study conducted in East Sussex, showed treatment with a garlic supplement, containing allicin, decreased the likelihood of catching a cold in more then half of the volunteers (Josling P. Preventing the common cold with a garlic supplement: a double-blind, placebo-controlled survey. Adv Ther. 2001 July-August; 18(4):189-93 (Abstract)), as well as a decrease the average duration of infections in those whom became afflicted.

Additionally, treatment of mice with allicin was shown to significantly reduced NF-κB binding to the nucleus (Bruck R, Aeed H, Brazovsky E, Noor T, Hershkoviz R. Allicin, the active component of garlic, prevents immune-mediated, concanavalin A-induced hepatic injury in mice. Liver Int. 2005 June; 25(3):613-21 (Abstract)). This reduction in binding was also accompanied with an inhibition of TNF-α mediated T cell adhesion to the extracellular matrix components as wells as to endothelial cells. It is herein understood by the inventors that the incorporation of an inhibitor of NF-κB-transcription binding and TNF-α mediated T cell adhesion, in an anti-inflammatory composition, will effectively reduce immune-mediated cellular damage.

In an embodiment of the present invention, which is set forth in greater detail in the examples below, the immune enhancing, anti-inflammatory composition comprises garlic. A serving of the immune enhancing, anti-inflammatory composition comprises from about 0.0001 g to about 1.0000 g g of garlic. The preferred dosage of a serving of the immune enhancing, anti-inflammatory composition comprises about 0.0010 g of an garlic.

### Cranberry Extract

Cranberries are the fruit from a species of evergreen dwarf scrubs found in acidic bogs throughout the cooler parts of the Northern Hemisphere. Cranberries are a major commercial crop in the United States and Canada where they are processed into consumer products, like juices and sauces. They are also a source of flavonoids, chemicals which are known to provide certain health benefits to the immune system. More specifically cranberries provide an abundant source of proanthocyanidins, which are polymers made from multiple anthocyanidin-like molecules known as flavanols. These are called proanthocyanidins because under acidic conditions they are broken apart to yield their constituent anthocyanidins. Proanthocyanidins are also known as leucoanthocyanins, leucodelphinins, leucocyanins, anthocyanogens, epicatechin-catechin polymers or procyanins.

Procyanidin B(2), epicatechin-epicatechin dimer, has been shown to strongly inhibit arachidonic acid inflammatory reactions (Chen D M, Cai X, Kwik-Uribe C L, Zeng R, Zhu X Z. Inhibitory effects of procyanidin B(2) dimer on lipid-laden macrophage formation. J Cardiovasc Pharmacol. 2006 Aug; 48(2):54-70 (Abstract)). Arachidonic acid is metabolized by the cells of the body to produce potent mediators of inflammation, leukotrienes and prostaglandins.

Leukotrienes are secreted lipid mediators of the inflammation response produced by leukocytes from arachidonic acid, which is a precursor for several mediators involved in inflammation. The production of leukotrienes is increased during inflammation and can produce and sustain the symptoms of inflammation (Hedqvist P, Gautam N, Lindbom L. Interactions between leukotrienes and other inflammatory mediators/modulators in the microvasculature. Am J Respir Crit Care Med. 2000 February; 161(2 Pt 2):S117-9). It is herein understood by the inventors that the incorporation of an inhibitor of leukotriene biosynthesis in an anti-inflammatory composition will effectively reduce inflammation observed as a result of infection or injury.

In an embodiment of the present invention, which is set forth in greater detail in the examples below, the immune enhancing, anti-inflammatory composition comprises an extract of cranberry. A serving of the immune enhancing, anti-inflammatory composition comprises from about 0.0001 g to about 1.0000 g of an extract of cranberry. The preferred dosage of a serving of the immune enhancing, anti-inflammatory composition comprises about 0.0010 g of an extract of cranberry.

In an embodiment of the present invention, which is set forth in greater detail Example 1, the immune enhancing, anti-inflammatory composition comprises vitamin C, an extract of Andrographis paniculata, vitamin A (Retinyl palmitate), an extract of blackberry, garlic, and an extract of cranberry. The composition is provided in any acceptable and suitable oral dosage form as known in the art to attenuate the intensity and duration of inflammatory reaction as a result of infection or injury, as well as reduce the immune-mediated cell damage associated with infections. The composition may also be provided in a prolonged or extended release format.

While, not wishing to be bound by theory, the present invention is comprised of components that have been shown to inhibit the binding of NF-κB to the promoter regions of specific target genes, thus reducing the expression of pro-inflammatory cytokines, such as TNF-α and IL-1. It is herein understood by the inventors that attenuation of the production of pro-inflammatory cytokines will result in decreased intensity and duration of inflammation-related symptoms.

Furthermore, the present invention comprises components that have been shown to lower the production of pro-inflammatory cytokines, such as IL-12 and TNF-α, while at the same time enhancing the production of the immune modulating cytokine, IL-10 (Wang X, Allen C, Ballow M. Retinoic Acid Enhances the Production of IL-10 While Reducing the Synthesis of IL-12 and TNF-alpha from LPS-Stimulated Monocytes/Macrophages. J Clin Immunol. 2007 Jan. 26; [Epub ahead of print] (Abstract)). It is herein understood by the inventors that limiting the production of pro-inflammatory mediators through various mechanisms while simultaneously increasing immune modulating cytokines, will result more controlled and less severe inflammatory response to injury and infection. The present invention additionally comprises components which are known to afford the body protection against reactive oxygen species. It is herein understood by the inventors that by reducing the severity of an inflammatory response and substantially simultaneously affording the body protection against byproducts of an immune response further cellular damage as a result of an insult will be reduced. The insult may be in the form of a physical insults, or a of a pathogenic nature.

Additionally, as the present invention comprises components that have been shown to significantly reduce the binding of the transcription factor NF-κB to the promoter region of specific target genes, as well as inhibition of TNF-α mediated T-cell adhesion to the extracellular matrix components and to endothelial cells, it is herein understood by the inventors that inhibition of NF-κB-binding and TNF-α mediated T-cell adhesion, will effectively reduce immune-mediated cell damage.

Further to the aforementioned functions, the present invention comprises components that inhibit arachidonic acid metabolization by the cells of the body to produce potent mediators of inflammation, such as leukotrienes and prostaglandins. It is herein understood by the inventors that this action will effectively reduce inflammation observed as a result of infection or injury.

The present invention is understood by the inventors to inhibit inflammation resulting from stimulation of the innate immune system of an individual by pathogens by substantially simultaneously affecting several mechanisms in the body pertaining to inflammation. Furthermore, the present invention affords the body of a user protection from byproducts of an immune-response which are known to be detrimental to the body of an afflicted mammal. The mechanisms are herein disclosed.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, an effervescent tablet, or as a dietary gel. The preferred dosage form of the present invention is as a capsule.

Furthermore, the dosage form of the nutritional supplement is provided in accordance with customary processing techniques for herbal and nutritional supplements in any of the forms mentioned above. Additionally, the nutritional supplement set forth in the example embodiment herein disclosed may contain any appropriate number and type of excipients, as is well known in the art.

Although the following example illustrates the practice of the present invention in one of its illustrative compositional embodiments, the example should not be construed as limiting the scope of the invention. Other embodiments will be apparent to one of skill in the art from consideration of the specifications and example.

### EXAMPLES

An immune enhancing, anti-inflammatory supplement comprising the following ingredients per serving is prepared for consumption as a effervescent tablet:

about 1.100 g of vitamin C, about 0.001 g of an extract of Andrographis paniculata, about 5010 IU of vitamin A (Retinyl palmitate), about 0.001 g of an extract of blackberry standardized flavonoids, about 0.001 g of garlic, and about 0.0010 g of an extract of cranberry standardized to proanthocyanidins.

### Extensions and Alternatives

In the foregoing specification, the invention has been described with a specific embodiment thereof; however, it will be evident that various modifications and changes may be made thereto without departing from the broader spirit and scope of the invention.

1. A composition comprising an effective amount of vitamin C and an extract of Andrographis Paniculata wherein the composition inhibits an inflammatory response and substantially simultaneously protects against immune response byproducts and maintains immune cell populations in a user.

2. The composition of claim 1 wherein the vitamin C and the extract of Andrographis Paniculata act synergistically through differing molecular mechanisms to inhibit inflammation and enhance immunity.

3. The composition of claim 2 wherein the extract of Andrographis paniculata inhibits the binding of the NF-κB transcription factor to DNA.

4. The composition of claim 2 wherein the vitamin C acts to increase the infiltration of leukocytes and simultaneously protect cells from reactive oxygen species produced during immune responses.

5-7. (canceled)

8. The composition of claim 1, further comprising an effective amount of garlic.

9. The composition of claim 8 wherein the garlic acts to inhibit TNF-α mediated T cell adhesion.

10. The composition of claim 9 wherein the inhibition of TNF-α mediated T cell adhesion effectively reduces immune-mediated cellular damage.

11-30. (canceled)

31. The composition of claim 1 wherein the composition is provided in an effervescent capsule.

32. (canceled)

33. The composition of claim 1 wherein the composition is provided in an orally acceptable dosage format selected from the group consisting of a tablet, a caplet, a dietary gel and effervescent tablet.

34. (canceled)
